SEQUENCE LISTING (11 sequences)

SEQ ID NO: 1 - DNA, 58 bp, Artificial Sequence
  Synthetic - shPIK3CA
ccggaatgaaagctcactctggattctcgagaatccagagtgagctttcatttttttg

SEQ ID NO: 2 - DNA, 58 bp, Artificial Sequence
  Synthetic - shPIK3CB
ccgggcgggagagtagaatatgtttctcgagaaacatattctactctcccgctttttg

SEQ ID NO: 3 - DNA, 58 bp, Artificial Sequence
  Synthetic - shPIK3CD
ccgggacccagaagtgaacgactttctcgagaaagtcgttcacttctgggtctttttg

SEQ ID NO: 4 - DNA, 58 bp, Artificial Sequence
  Synthetic - shPIK3CG_1
ccgggcagagcttcttcaccaagatctcgagatcttggtgaagaagctctgctttttg

SEQ ID NO: 5 - DNA, 58 bp, Artificial Sequence
  Synthetic - shPIK3CG_2
ccgggccctatcaaatgaaacaattctcgagaattgtttcatttgatagggctttttg

SEQ ID NO: 6 - DNA, 59 bp, Artificial Sequence
  Synthetic - shPIK3CG 3′UTR
ccgggccttatccatttcccatttactcgagtaaatgggaaatggataaggcttttttg

SEQ ID NO: 7 - DNA, 58 bp, Artificial Sequence
  Synthetic - shPIK3R1
ccggccttcagttctgtggttgaatctcgagattcaaccacagaactgaaggtttttg

SEQ ID NO: 8 - DNA, 58 bp, Artificial Sequence
  Synthetic - shPIK3R2
ccgggcagatgaagcgtactgcaatctcgagattgcagtacgcttcatctgctttttg

SEQ ID NO: 9 - DNA, 58 bp, Artificial Sequence
  Synthetic - shPIK3R3
ccgggctttggacaaccgagaaatactcgagtatttctcggttgtccaaagctttttg

SEQ ID NO: 10 - DNA, 58 bp, Artificial Sequence
  Synthetic - shPIK3R5
ccggcaggatctataaactcttcaactcgagttgaagagtttatagatcctgtttttg

SEQ ID NO: 11 - DNA, 58 bp, Artificial Sequence
  Synthetic - shPIK3R6
ccggccagatctacacagtcaagatctcgagatcttgactgtgtagatctggtttttg

(19) United States
(12) Patent Application Publication
(10) Pub. No.: US 2023/0201202 A1
(43) Pub. Date: Jun. 29, 2023

(54) COMPOSITIONS AND METHODS FOR SENSITIZING ACUTE MYELOID LEUKEMIAS TO CHEMOTHERAPY

(71) Applicant: Duke University, Durham, NC (US)

(72) Inventors: Kris WOOD, Durham, NC (US); Kevin H. Lin, Durham, NC (US); Justine Rutter, Durham, NC (US); Xiaohui Hazel ANG, Durham, NC (US)

(21) Appl. No.: 17/927,575

(22) PCT Filed: May 26, 2021
(86) PCT No.: PCT/US21/34255

§ 371 Date: Nov. 23, 2022

Related U.S. Application Data
(60) Provisional application No. 63/030,338, filed on May 27, 2020.

(51) Int. Cl. A61K 31/519; A61K 31/427; A61K 31/498; A61K 45/06

(52) U.S. Cl. CPC A61K 31/519; A61K 31/427; A61K 31/498; A61K 45/06

(57) ABSTRACT

The present invention provides methods for reducing the viability of cells that express PI3Kγ, treating myeloid malignancies, and sensitizing cells, particularly cancer cells that express PI3Kγ to chemotherapeutic agents. The methods comprise contacting a cell or administering to the subject a PI3K inhibitor that inhibits PI3Kγ in an isoform-specific manner.

### CROSS-REFERENCE TO RELATED APPLICATIONS

This application claims priority to U.S. Provisional Application No. 63/030,338 filed on May 27, 2020, the contents of which are incorporated by reference in their entireties.

### STATEMENT REGARDING FEDERALLY SPONSORED RESEARCH

This invention was made with Government support under Federal Grant no. R01CA207083 awarded by the National Cancer Institute. The Federal Government has certain rights to this invention.

### SEQUENCE LISTING

A Sequence Listing accompanies this application and is submitted as an ASCII text file of the sequence listing named “155554_00606_ST25.txt” which is 2.41 KB in size and was created on May 20, 2021. The sequence listing is electronically submitted via EFS-Web with the application and is incorporated herein by reference in its entirety.

### BACKGROUND

Class I PI3K lipid kinases integrate diverse environmental stimuli by phosphorylating phosphatidylinositol 4,5-bisphosphate (PtdIns-4,5P₂) to phosphatidylinositol (3,4,5)-trisphosphate (PtdIns-3,4,5P₃), initiating a cascade of coordinated signaling events that together drive major oncogenic programs in human cancer [1, 2]. The PI3K pathway is one of the most frequently altered pathways in malignancy. Gain-of-function mutations and amplifications in genes that encode catalytic isoforms of PI3K (i.e., PIK3CA and PIK3CB) and deletions in PTEN (a major negative regulator of the pathway) are common oncogenic events across many tumor types [3, 4]. Consequently, most efforts to pharmacologically suppress the PI3K pathway have focused on targeting PI3K in patients with stable alterations in the pathway [5-7]. This has led to the development of pan-PI3K inhibitors [8, 9], isoform-selective PI3K inhibitors [7, 10-12], and inhibitors of PI3K effectors [3, 13]. Unfortunately, the clinical advancement of these agents has been stymied by dose-limiting toxicities arising from on-target suppression of growth factor signaling and nutrient utilization in non-malignant tissues [14-16]. These limitations can be minimized by targeting cancer dependencies driven by proteins with tissue- or tumor-restricted expression. Accordingly, there is a great need for novel compositions and therapies that target such proteins.

### SUMMARY

In a first aspect, the present invention provides methods of reducing the viability of a cell that expresses PI3Kγ in a subject. The methods comprise contacting a cell that expresses PI3Kγ with an effective amount of a PI3Kγ inhibitor or PI3Kγ degradation molecule.

In a second aspect, the present invention provides methods of treating a myeloid malignancy in a subject. The methods comprise administering to the subject a therapeutically effective amount of a PI3Kγ inhibitor or PI3Kγ degradation molecule.

In a third aspect, the present invention provides methods of sensitizing a cell that expresses PI3Kγ in a subject to a chemotherapeutic agent. The methods comprise administering to the subject a therapeutically effective amount of a PI3Kγ inhibitor or PI3Kγ degradation molecule and a therapeutically effective amount of a chemotherapeutic agent.

### BRIEF DESCRIPTION OF THE DRAWINGS

FIG. 1 demonstrates that p110γ is an isoform-specific regulator of AKT in AML. (a,b) Expression of catalytic (a) and regulatory (b) Class I PI3K isoforms in normal and tumor tissue samples. Tumor expression data from TCGA database; normal expression data from GeTex expression database. Data accessed through Gepia gene expression portal. (c) Immunoblot depicting effect of doxycycline(dox)-induced shRNA-mediated knockdown of catalytic PI3K isoforms on expression of phosphorylated AKT in OCI-AML2 cells. Cells treated for 72 hours with 75 ng/mL of dox prior to collection. (d) Immunoblot depicting effect of MK-2206 (AKT inhibitor), IPI-549 (p110γ inhibitor) and BYL-719 (p110α inhibitor) treatment on phosphorylation of AKT and downstream substrates, BAD and GSK3B in OCI-AML2 cells. Cells treated with indicated nM concentrations of drug. (e) Immunoblot depicting effect of wild-type PIK3CG overexpression in OCI-AML2 cells on phosphorylation of AKT and downstream substrate, TSC2, with and without a 3′ UTR targeting shRNA against PIK3CG. (f) Immunoblot depicting effect of GPCR-G-protein uncoupling by pertussis toxin (Ptx) on phosphorylation of ERK, AKT and downstream AKT substrate, TSC2, in a panel of AML cell lines. Cells treated with Ptx (100 ng/mL) for 24 hours. (g) Immunoblot depicting effect of stromal cell conditioned media on phosphorylation of AKT and downstream substrate, TSC2, with and without IPI-549 treatment. Serum-free media was conditioned by human BM-MSCs (HS-5 cell line) and applied to AML cells for 1.5 hours prior to collection. (h) Immunoblot depicting effect of CXCL12 cytokine stimulation on phosphorylation of AKT with and without IPI-549 treatment. Cells were treated for 1 hour with IPI-549 and stimulated with 200 ng/mL SDF1α for 30 minutes prior to collection. g,h HL-60 and OCI-AML3 cells were treated with 500 nM and 100 nM of IPI-549, respectively.

FIG. 2 demonstrates that p110γ inhibition potentiates the cytotoxicity of chemotherapies used in AML. (a) DepMap CRISPR/Cas9 dependency profiling data depicting essentiality of the four catalytic isoforms of PI3K. Data are ordered by unsupervised hierarchical clustering of Min-Max normalized averaged dependencies across cell lines in a given cancer type. Only cancer types with >5 cell lines were included in analysis. (b) Effect of shRNA-mediated depletion of PIK3CA, PIK3CB, PIK3CD or PIK3CG on cell viability in OCI-AML2 cells. Cells treated with 75 ng/mL of dox for 7 days and then assessed for viability using Cell Titer Glo. Viability for each dox-induced shRNA knockdown was normalized to viability of cells treated without dox. (c) Pooled drug-modifier screens conducted in the presence of IPI-549 or MK-2206. OCI-AML2 cells were transduced with a full-genome CRISPR/Cas9 library (n=4 sgRNA constructs targeting each gene) at a low multiplicity of infection (MOI) of 0.2, selected with puromycin and separated into treatment conditions (n=2 replicates per condition). After 12 days of drug exposure, the relative abundance of each sgRNA was compared between the drug-treated and vehicle-treated populations. Gene-level scores were obtained by averaging sgRNA-level comparisons. Spearmen coefficient (p) calculated to assess correlation between the IPI-549 and MK-2206 gene scores. Genes with a log 2 depletion of <−0.5 or enrichment of >0.5 designated in orange and blue, respectively. Non-targeting control genes depicted as white points. (d) Effect of IPI-549 or MK-2206 in combination with ABT-199 across a panel of AML cell lines. GI₅₀ value of combination treatment compared to ABT-199 treatment alone. (e) Effect of IPI-549 or BYL-549 in combination with daunorubicin across a panel of AML cell lines. GI₅₀ value of combination treatment compared to daunorubicin treatment alone. (f) Effect of shRNA-mediated depletion of PIK3CA, PIK3CB, PIK3CD or PIK3CG on daunorubicin sensitivity in OCI-AML2 cells. Cells treated with daunorubicin (15 nM) and 75 ng/mL of dox for 7 days and analyzed as in (a).

FIG. 3 demonstrates that selective p110γ inhibition potentiates the effects of certain cytotoxic and targeted chemotherapies. (a) Effect of IPI-549 on sensitivity to doxorubicin, idarubicin, 563845, JQ1, and etoposide (top left), selumetinib and SCH772984 (top right), and sorafenib, midostaurin, gilteritinib, and quizartinib (bottom left) in indicated AML cell lines. GI₅₀ value of combination treatment compared to indicated drug treatment alone. (b) Effect of IPI-549, BYL-719, TGX-221, or CAL-101 on sensitivity to doxorubicin or S63845 (top) or selinexor (bottom) in indicated AML cell lines. GI₅₀ value of combination treatment compared to indicated drug treatment alone. (c) Effect of structurally distinct, p110γ-selective inhibitors AS252424 or AS605240 on sensitivity to doxorubicin, ABT199, or S63845 in OCI-AML2 cells.

FIG. 4 is an extension of FIG. 1. (a) Immunoblot depicting effect of dox-induced shRNA-mediated knockdown of PIK3CA and PIK3CG on expression of phosphorylated AKT in OCI-AML3 cells. (b) Immunoblot depicting effect of BYL-719 (p110α inhibitor) and IPI-549 (p110γ inhibitor) on phosphorylation of AKT in a panel of AML cell lines following 1 hour of drug treatment. OCI-AML2 and cells were treated with 500 nM of BYL-719 and IPI-549; MV4; 11, MOLM-13, THP-1 and OCI-AML3 cells were treated with 200 nM of BYL-719 and IPI-549. (c) Immunoblot depicting effect of dox-induced shRNA-mediated knockdown of regulatory PI3K isoforms on expression of phosphorylated AKT in OCI-AML2 cells. (d) Fold expression of indicated regulatory PI3K isoforms in OCI-AML2 cells containing dox-inducible shRNAs. Data are mean±SEM for n=3 biologically independent experiments. P-values computed by two-sided two-sample t-Test for equal means. a,c,d, Cells treated for 72 hours with 75 ng/mL of dox prior to collection.

FIG. 5 is an extension of FIG. 2. (a) Effect of shRNA-mediated depletion of PIK3CA or PIK3CG on cell viability in OCI-AML3 cells. Cells treated with 75 ng/mL of dox for 7 days and then assessed for viability using Cell Titer Glo. Viability for each dox-induced shRNA knockdown was normalized to viability of cells treated without dox. (b,c) GI₅₀ values of IPI-549 (b) and MK-2206 (c) in 10 cell line panel. Cell lines ranked by GI₅₀ value. (d) Correlation between log 2-transformed GI₅₀ values of IPI-549 and MK-2206 across 11 cell line panel. Spearmen correlation coefficient (p) and p-value provided to demonstrate correlation and significance. Dashed line depicts linear regression of values. (e) Correlation between gene dependency scores of AKT1 and AKT2 versus PIK3CG in a panel of 15 AML cell lines. Data from DepMap CRISPR/Cas9 dependency profiling. Pearson correlation coefficient (p) and p-value provided to demonstrate correlation and significance. (f) Correlation between gene dependency scores of AKT1 and AKT2 versus PIK3CG across all cell lines from in DepMap CRISPR/Cas9 dependency profiling dataset. Pearson correlation coefficient (p) and p-value provided to demonstrate correlation and significance. (g) Effect of IPI-549 or MK-2206 in combination with MLN-128 across a panel of AML cell lines. GI₅₀ value of combination treatment compared to MLN-128 treatment alone. (h) Effect of shRNA-mediated depletion of PIK3CA or PIK3CG on cell viability in OCI-AML3 cells. Cells treated with daunorubicin (10 nM) and 75 ng/mL of dox for 7 days and analyzed as in (a). (i) Kaplan-Meier curve for overall survival of patients with whose leukemic blasts expressed high (upper 33%) or low (bottom 33%) expression of PIK3R5. Data from TCGA (n=75 AML patients represented in each group). Dashed lines indicate median overall survival. a-c, e, h Data are mean±SEM for n=3 biologically independent experiments. P-values computed by two-sided two-sample t-Test for equal means.

FIG. 6 demonstrates that chemosensitization mediated by PI3Kγ inhibition with IPI-549 is not observed with all AML drugs. Specifically, it was not observed with cytarabine, azacytidine, decitabine, methotrexate, glasdegib, docetaxel, and oxaliplatin. Data shown from OCI-AML2 cells.

### DETAILED DESCRIPTION

The present invention provides methods for reducing the viability of cells that express PI3Kγ (e.g., myeloid cells, among others), treating myeloid malignancies, and sensitizing cells that express PI3Kγ to chemotherapeutic agents.

This disclosure is based on the inventors' discovery that in some hematological malignancies, such as acute myeloid leukemia (AML), suppression of a myeloid-restricted PI3K isoform of PI3K (i.e., PI3Kγ) blocks PI3K/AKT signaling, compromises cell fitness, and sensitizes cells to chemotherapy. Therefore, targeted inhibition of PI3Kγ signaling can be used to inhibit PI3K signaling selectively in myeloid malignancies (e.g., AML) to enhance the efficacy of chemotherapies while avoiding the toxicities that have historically limited clinical inhibition of PI3K.

### Methods

In a first aspect, the present invention provides methods of reducing the viability of a cell that expresses PI3Kγ in a subject. The methods comprise contacting a cell that expresses PI3Kγ, e.g., a myeloid cell, with an effective amount of a PI3Kγ inhibitor or PI3Kγ degradation molecule. After contact, the cell may suitably undergo cell cycle arrest/slowing, apoptosis, or another form of cell death. In some embodiments, the cell is in vivo in a subject in need of such treatment, for example, in a subject having a myeloid malignancy. The methods comprise administering to the subject a therapeutically effective amount of a PI3Kγ inhibitor or PI3Kγ degradation molecule. In some embodiments, the cell is a hematopoietic cell, a myeloid progenitor cell, or a myeloid cell. In one preferred embodiment, the cell is a myeloid cell. Method of ascertaining if a cell expresses PI3Kγ are readily known and well understood by one of ordinary skill in the art.

The methods described herein may use a PI3Kγ inhibitor or a PI3Kγ degradation molecule and any method described herein for use of a PI3Kγ inhibitor can suitable use a PI3Kγ degradation molecule and believed to have similar effect. The term “PI3Kγ degradation molecule” refers to molecules or drugs that are able to trigger the degradation of PI3Kγ enzyme in an isoform specific manner. Suitable PI3Kγ degradation molecule can be determined by one skilled in the art, for example, molecular glues and/or proteolysis targeting chimera (PROTACs) that specifically target PI3Kγ.

PI3K inhibitors are a class of drugs that function by inhibiting one or more of the PI3K enzymes. PI3K enzymes are members of a conserved family of intracellular lipid kinases that phosphorylate the 3′—OH group on phosphatidylinositols or phosphoinositides. The PI3K family includes kinases with distinct substrate specificities, expression patterns, and modes of regulation. The class I PI3Ks (i.e., p110α, p110β, p110γ, and p110δ) are typically activated by tyrosine kinases or G-protein coupled receptors to generate PIP3, which engages downstream mediators such as those in the AKT/PDK1 pathway, mTOR, the Tec family kinases, and the Rho family GTPases. The class II PI3Ks (i.e., PI3K-C2α, PI3K-C2β, PI3K-C2γ) and III PI3Ks (i.e., Vps34) play a key role in intracellular trafficking through the synthesis of PI(3)P and PI(3,4)P2. The class I PI3Ks comprise a p110 catalytic subunit and a regulatory adapter subunit. Four isoforms of the p110 subunit (i.e., PI3K-α (alpha), PI3K-β (beta), PI3K-γ (gamma), and PI3K-δ (delta)) have been implicated in various biological functions. Class I PI3Kα is involved, for example, in insulin signaling, and has been found to be mutated in solid tumors. Class I PI3Kβ is involved, for example, in platelet activation and insulin signaling. Class I PI3Kγ plays a role in mast cell activation, innate immune function, and immune cell trafficking (chemokines). Class I PI3Kδ is involved, for example, in B-cell and T-cell activation and function and in Fc receptor signaling in mast cells.

Any PI3K inhibitor that specifically inhibits PI3Kγ in an isoform-specific manner may be used in the methods of the present invention. Advantageously, the PI3K inhibitor is capable of inhibiting p110γ-p101 complex signaling. Suitable PI3Kγ inhibitors include, without limitation, IPI-549 ((S)-2-Amino-N-(1-(84(1-methyl-1H-pyrazol-4-yl)ethynyl)-1-oxo-2-phenyl-1,2-dihydroisoquinolin-3-yl)ethyl)pyrazolo[1,5-a]pyrimidine-3-carboxamide), AS252424 ((5Z)-5-[[5-(4-fluoro-2-hydroxyphenyl)furan-2-yl]methylidene]-1,3-thiazolidine-2,4-dione), AS605240 ((5Z)-5-(quinoxalin-6-ylmethylidene)-1,3-thiazolidine-2,4-dione), AS604850 ((5E)-5-[(2,2-Difluoro-1,3-benzodioxol-5-YL)methylene]-1,3-thiazolidine-2,4-dione), AZ1, AZ2, AZ3, AZ4, and NVS-PI3-4 (See, e.g., (See, e.g., PMID 30718815 and 29852070, Gangadhara, G., Dahl, G., Bohnacker, T. et al. A class of highly selective inhibitors bind to an active state of PI3Kγ. Nat Chem Biol 15, 348-357 (2019). and Pemberton et al., J. Med. Chem. 2018, 61, 12, 5435-5441, Publication Date: May 31, 2018, PMID 23683463, incorporated by reference in its entirety, e.g., N-(5-{2-[(1S)-1-Cyclopropylethyl]-7-(methylsulfonyl)-1-oxo-2,3-dihydro-1H-isoindol-5-yl}-4-methyl-1,3-thiazol-2-yl)acetamide; N-(5-{2-[(1 S)-1-Cyclopropylethyl]-7-(methylsulfamoyl)-1-oxo-2,3-dihydro-1H-isoindol-5-yl}-4-methyl-1,3-thiazol-2-yl)acetamide; and N-(5-{2-[(1S)-1-Cyclopropylethyl]-7-[(methylsulfonyl)amino]-1-oxo-2,3-dihydro-1H-isoindol-5-yl}-4-methyl-1,3-thiazol-2-yl)acetamide, among others).

The methods of the present invention can be used to reduce the viability of a cell that expresses PI3Kγ as compared to untreated cells. Cell viability can be quantified using any appropriate assay known in the art. Cell viability assays often quantify markers of metabolically active (living) cells or markers of cell death/cytostasis. For example, ATP levels, the ability to reduce a substrate, and enzymatic/protease activity can be measured as indicators of metabolic activity, while chromosomal degradation and Annexin V can be measured as indicators of cell death. Cell viability assays may involve staining or immunoblotting, or they may be performed using commercially available kits, such at the CellTiter-Glo® Luminescent Cell Viability Assay kit (Promega). Cell viability may also be assessed using cell counting.

In one embodiment, the present invention can be used to reduce the viability of a myeloid cells, specifically a myeloid malignant cell or myeloid cancer cell. The method may result in the reduction in the number of myeloid cancer cells, reduction in growth the myeloid cancer cells, or a reduction in the proliferation of the myeloid cancer cells.

In a second aspect, the present invention provides methods of treating a hematologic disorder in a subject, preferably a myeloid disorder or malignancy. The methods comprise administering to a subject a therapeutically effective amount of a PI3Kγ inhibitor such that the hematologic disorder is treated in the subject. In a preferable embodiment, the hematologic disorder is a myeloid malignancy.

In some embodiments, the methods comprise treating a myeloid malignancy in a subject by administering an effective amount of the PI3Kγ inhibitor. The myeloid malignancy comprises cells that express PI3Kγ, and therefore are susceptible to PI3Kγ inhibition. In some embodiments, the myeloid malignancy is acute myeloid leukemia (AML).

As used herein, “treating” or “treatment” describes the management and care of a subject for the purpose of combating a disease, condition, or disorder. Treating includes administering a treatment to prevent the onset of the symptoms or complications, to alleviate the symptoms or complications, or to eliminate the disease, condition, or disorder. For example, treating cancer in a subject includes the reducing, repressing, delaying or preventing of cancer growth, reduction of tumor volume, and/or preventing, repressing, delaying or reducing metastasis of the tumor. Treating cancer in a subject also includes the reduction of the number of tumor cells within the subject.

In some embodiments, the methods further comprise administering a therapeutically effective amount of a chemotherapeutic agent. In some embodiments, the myeloid malignancy has developed some level of resistance to the chemotherapeutic agent, and co-administration with the PI3Kγ inhibitor allows the chemotherapeutic agent to continue to be effective.

As used herein, “subject” or “patient” refers to mammals and non-mammals. A “mammal” may be any member of the class Mammalia including, but not limited to, humans, non-human primates (e.g., chimpanzees, other apes, and monkey species), farm animals (e.g., cattle, horses, sheep, goats, and swine), domestic animals (e.g., rabbits, dogs, and cats), or laboratory animals including rodents (e.g., rats, mice, and guinea pigs). Examples of non-mammals include, but are not limited to, birds, and the like. The term “subject” does not denote a particular age or sex. In one specific embodiment, a subject is a mammal, preferably a human. In some embodiments, the subject is suffering from a hematologic malignancy. In some embodiments, the hematologic malignancy is a myeloid malignancy. In certain embodiments, the hematologic or myeloid malignancy is AML.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intraaural administration, intracerebral administration, rectal administration, sublingual administration, buccal administration, and parenteral administration, including injectable such as intravenous administration, intra-arterial administration, intramuscular administration, intradermal administration, intrathecal administration, and subcutaneous administration. Administration can be continuous or intermittent. Local administration is also contemplated, e.g., to the bone marrow of cells

The terms “effective amount” or “therapeutically effective amount” refer to an amount sufficient to effect beneficial or desirable biological or clinical results. That result can be reducing, alleviating, inhibiting or preventing one or more symptoms of a disease or condition, reducing, inhibiting or preventing the growth of cancer cells, reducing, inhibiting or preventing metastasis of the cancer cells or invasiveness of the cancer cells or metastasis, or reducing, alleviating, inhibiting or preventing one or more symptoms of the cancer or metastasis thereof, or any other desired alteration of a biological system. In some embodiments, the effective amount is an amount suitable to provide the desired effect, e.g., an anti-tumor response. An anti-tumor response may be demonstrated, for example, by a decrease in tumor size, tumor growth, or an increase in immune cell activation (e.g., CD8+ T cell activation).

Methods for determining an effective means of administration and dosage are well known to those of skill in the art and will vary with the formulation used for therapy, the purpose of the therapy, the target cell being treated, and the subject being treated. Single or multiple administrations can be carried out with the dose level and pattern being selected by the treating physician.

In some embodiments, the subject has a myeloid malignancy. “Myeloid malignancies” are a heterogeneous group of clonal disorders that are characterized by excessive proliferation, abnormal self-renewal, and/or differentiation defects of hematopoietic cells and myeloid progenitor cells. Myeloid malignancies include “chronic stages” such as myeloproliferative neoplasms (MPNs), myelodysplastic syndromes (MDS), and chronic myelomonocytic leukemia (CMML) as well as “acute stages”, i.e, acute myeloid leukemia (AML). AML can occur de novo or follow a chronic stage (secondary AML). In some embodiments, the myeloid malignancy is a myeloproliferative neoplasm (MPN) or a myelodysplastic syndrome (MDS). MPNs are a group of blood cancers in which excess blood cells (i.e., red blood cells, white blood cells, or platelets) are produced in the bone marrow. In MPNs, the neoplasm (abnormal growth) starts out as benign and can later become malignant. There are several subcategories of MPNs, including chronic myeloid leukemia, chronic neutrophilic leukemia, polycythemia vera, primary myelofibrosis, essential thrombocythemia, chronic eosinophilic leukemia, and mastocytosis. MDS are a group of cancers in which immature blood cells in the bone marrow fail to mature into healthy blood cells. MDS may progress to leukemia. In other embodiments, the myeloid malignancy is acute myeloid leukemia (AML). AML is a cancer of the myeloid line of blood cells. AML is characterized by the rapid growth of abnormal cells that build up in the bone marrow and blood and interfere with normal blood cell production.

In the Examples, the inventors demonstrate that PI3Kγ inhibitors can be used to sensitize AML to chemotherapeutic agents, particularly apoptotic chemotherapeutic agents. Thus, in some embodiments, the methods further comprise administering to the subject a therapeutically effective amount of a chemotherapeutic agent in combination with the PI3Kγ inhibitor. In these embodiments, the apoptotic chemotherapeutic agent may be co-administered with the PI3Kγ inhibitor. The term “co-administration” encompasses administration of two or more agents to subject so that both agents and/or their metabolites are present in the subject at the same time. Co-administration includes simultaneous administration in separate compositions, administration at different times in separate compositions, or administration in a single composition in which both agents are present.

A “chemotherapeutic agent”, “anti-cancer agent”, or “anti-tumor agent” refers to any agent useful in the treatment of a neoplastic condition. Particularly, the methods of the present invention provide enhancement of proapoptotic chemotherapeutic agents, i.e. agents that result in the apoptosis of the cell. Suitable pro apoptotic chemotherapeutic agents are known and understood by one skilled in the art.

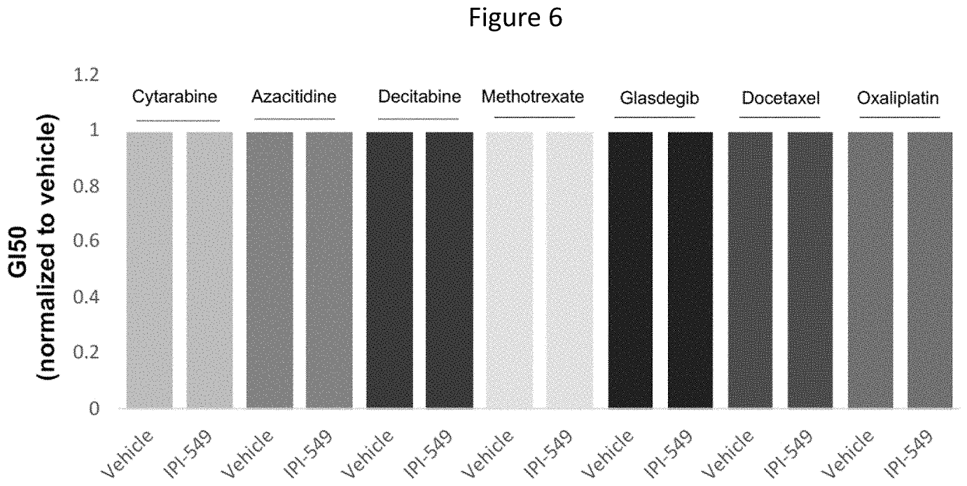

The inventors surprisingly discovered the ability of PI3Kγ to chemosensitize cancer cells to treatment with a chemotherapeutic agent. As demonstrated in the Examples, the chemosensitization mediated by PI3Kγ inhibition does not occur with all AML drugs (FIG. 6). Without wishing to be bound by theory, the inventors hypothesize that the PI3Kγ inhibition contributes to apoptotic priming, i.e., the process by which a cell is moved from a pro-survival state to a pro-apoptotic state by the activation of apoptotic pathways. Thus, the inventors hypothesize that PI3Kγ inhibition is particularly effective at sensitizing cells to chemotherapeutic agents that are proapoptotic, i.e., chemotherapeutic agents that promote or cause apoptosis. Therefore, in some embodiments, the chemotherapeutic agent is proapoptotic.

In some embodiments, the chemotherapeutic agent is selected from the group consisting of: a BCL2-inhibitor (e.g., ABT-199), an mTOR inhibitor (e.g., MLN-128), an anthracycline (e.g., daunorubicin, doxorubicin, idarubicin), a MCL-1 inhibitor (e.g., S63845), a FLT-3 inhibitor (e.g., sorafenib, midostaurin, gilteritinib, quizartinib), an MEK1/2 inhibitor or ERK1/2 inhibitor (e.g., selumetinib, SCH772984), a topoisomerase inhibitor (e.g., etoposide), a BET inhibitor (e.g., JQ1), or a XPO1/CRM1 inhibitor (e.g., selinexor). In some embodiments, the chemotherapeutic agent is selected from the group consisting of: ABT-199, MLN-128, daunorubicin, doxorubicin, idarubicin, 563845, sorafenib, midostaurin, gilteritinib, quizartinib, selumetinib, SCH772984, etoposide, JQ1, and selinexor.

Further examples of chemotherapeutic agents include, but are not limited to, one or more of: an HDAC inhibitor, such as, e.g., belinostat, vorinostat, panobinostat, or romidepsin; an mTOR inhibitor, such as, e.g., everolimus (RAD 001); a proteasome inhibitor, such as, e.g., bortezomib or carfilzomib; a JAK inhibitor or a JAK/STAT inhibitor, such as, e.g., Tofacitinib, INCB16562, or AZD1480; a BCL-2 inhibitor, such as, e.g., ABT-737, ABT-263, or Navitoclax; a MEK inhibitor, such as, e.g., AZD8330 or ARRY-424704; an anti-folate, such as, e.g., pralatrexate; a famesyl transferase inhibitor, such as, e.g., tipifarnib; an antibody or a biologic agent, such as, e.g., alemtuzumab, rituximab, ofatumumab, or brentuximab vedotin (SGN-035); an antibody-drug conjugate, such as, e.g., inotuzumab ozogamicin, or brentuximab vedotin; a cytotoxic agent, such as, e.g., bendamustine, gemcitabine, oxaliplatin, cyclophosphamide, vincristine, vinblastine, anthracycline (e.g., daunorubicin or daunomycin, doxorubicin, or actinomycin or dactinomycin), bleomycin, clofarabine, nelarabine, cladribine, asparaginase, methotrexate, or pralatrexate; or other anti-cancer agents or chemotherapeutic agents, such as, e.g., fludarabine, ibrutinib, fostamatinib, lenalidomide, thalidomide, rituximab, cyclophosphamide, doxorubicin, vincristine, or R-CHOP (Rituximab, Cyclophosphamide, Doxorubicin or Hydroxydaunomycin, Vincristine or Oncovin).

In a third aspect, the method comprises, consists of, or consists essentially of administering to a subject suffering from a hematological disorder a therapeutically effective amount of a PI3K inhibitor such that the hematological disorder is sensitized to a standard treatment regimen.

In some embodiments, the methods involve sensitizing a cell that expresses PI3Kγ in a subject to a chemotherapeutic agent. The methods comprise administering to the subject a therapeutically effective amount of a PI3Kγ inhibitor and a therapeutically effective amount of a chemotherapeutic agent, preferably a proapoptotic chemotherapeutic agent as described above. In some embodiments, the cell is a hematopoietic cell, a myeloid progenitor cell, or a myeloid cell.

As used herein, the term “sensitizing” refers to the ability of the PI3Kγ inhibitor to enhance the sensitivity of the cell to a chemotherapeutic agent relative to its sensitivity to the chemotherapeutic agent in the absence of the PI3Kγ inhibitor.

The methods of the present invention specifically target the PI3K isoform PI3Kγ. To be effective, the methods should be applied to cells that express PI3Kγ. Thus, in some embodiments, the method further comprise determining whether the cell (e.g. cancer cell) expresses PI3Kγ prior to the administration step. PI3Kγ expression can be detected using conventional methods known in the art, including immunoassays assays, such as ELISA, western blotting, and flow cytometry; chromatographic methods; and protein mass spectrometry assays. Antibodies that bind to PI3Kγ are known in the art and some are commercially available. Alternatively, PI3Kγ expression can be detected at the RNA level using, for example, quantitative reverse transcription PCR (RT-qPCR) or RNA sequencing.

In some embodiments, the cell is associated with a myeloid malignancy. In some embodiments, the myeloid malignancy is a myeloproliferative neoplasm (MPN) or a myelodysplastic syndrome (MDS). In other embodiments, the myeloid malignancy is acute myeloid leukemia (AML).

In some embodiments, the chemotherapeutic agent is proapoptotic. In some embodiments, the chemotherapeutic agent is selected from the group consisting of: a BCL2-inhibitor (e.g., ABT-199), an mTOR inhibitor (e.g., MLN-128), an anthracycline (e.g., daunorubicin, doxorubicin, idarubicin), a MCL-1 inhibitor (e.g., S63845), a FLT-3 inhibitor (e.g., sorafenib, midostaurin, gilteritinib, quizartinib), an MEK1/2 inhibitor or ERK1/2 inhibitor (e.g., selumetinib, SCH772984), a topoisomerase inhibitor (e.g., etoposide), a BET inhibitor (e.g., JQ1), or a XPO1/CRM1 inhibitor (e.g., selinexor).

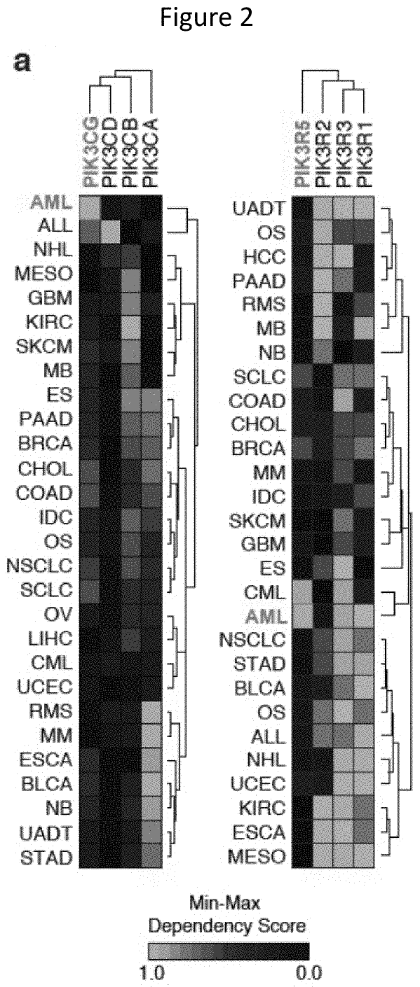

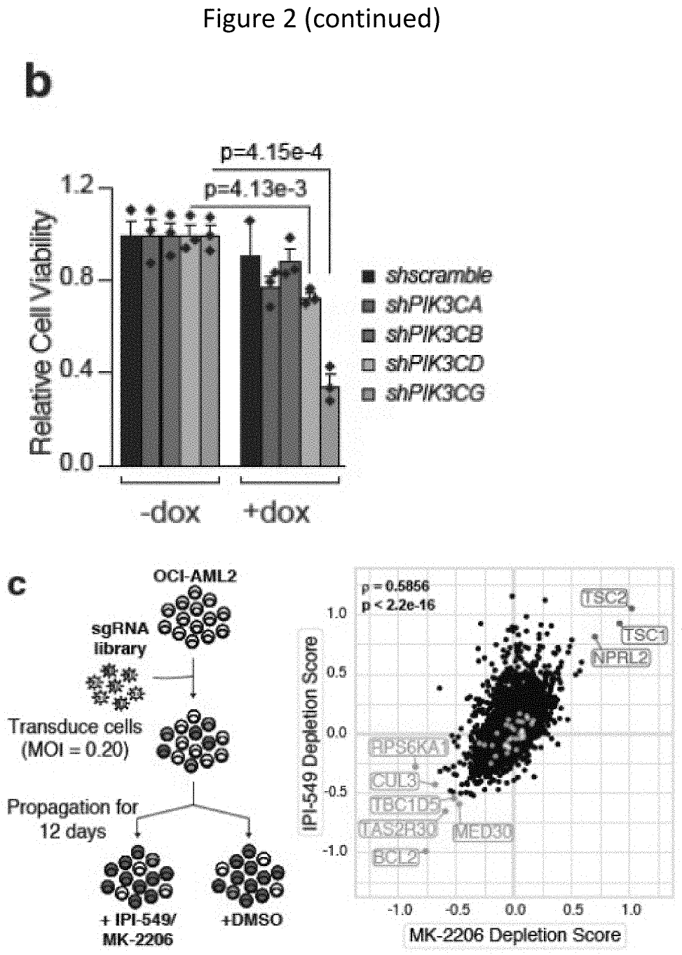

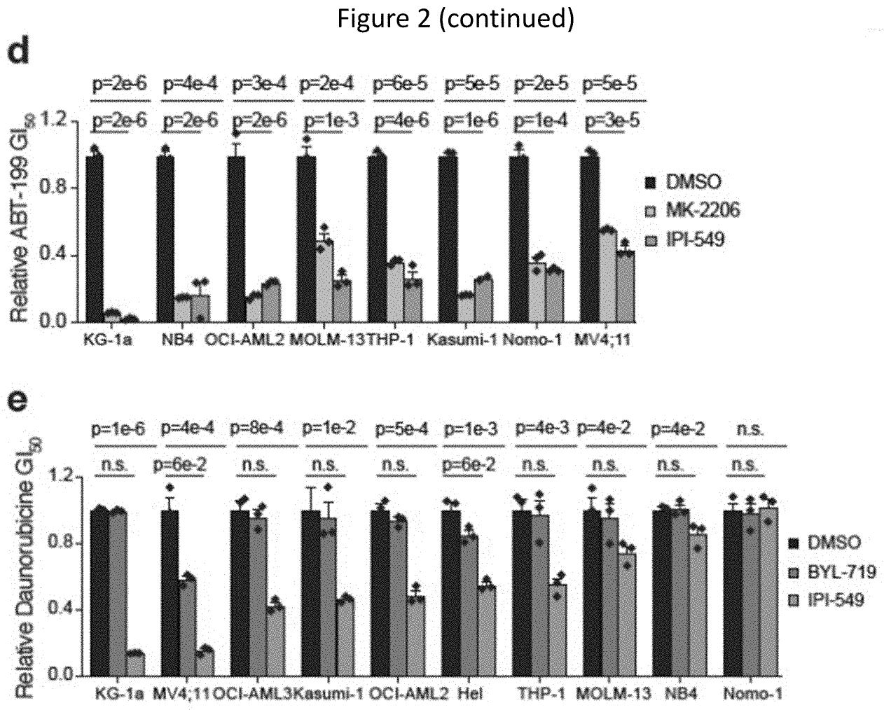

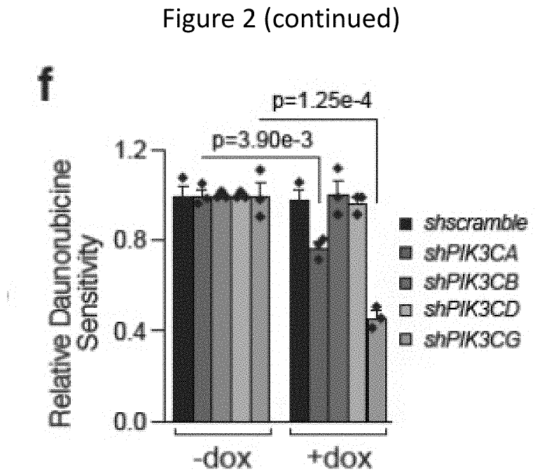

While the methods of the present invention are predominantly directed to cells associated with myeloid malignancies, the inventors envision that PI3Kγ inhibitors may also be useful for the treatment of other cancerous cells that express PI3Kγ. In FIG. 2a, the inventors demonstrate that the PIK3CG isoform is most essential in AML and secondarily essentially in acute lymphocytic leukemia (ALL), but is minimally essential in malignancies of non-hematopoietic origin. Thus, in another aspect, the present provides methods of treating and/or preventing a hematologic disorder in a subject. The methods comprise, consist of, or consist essentially of administering to a subject a therapeutically effective amount of a PI3Kγ inhibitor, or a pharmaceutical composition thereof. In another embodiment, the present disclosure provides a method of treating a disorder associated with proliferation of cells, wherein the cells express PI3Kγ, for example, a cancer cell.

In some embodiments, the hematologic disorder is selected from the group consisting of a myeloid disorder, lymphoid disorder, leukemia, lymphoma, myelodysplastic syndrome (MDS), myeloproliferative disease (MPD), mast cell disorder, and myeloma (e.g., multiple myeloma). In one embodiment, the blood disorder or the hematologic malignancy includes, but is not limited to, acute lymphoblastic leukemia (ALL), T-cell ALL (T-ALL), B-cell ALL (B-ALL), acute myeloid leukemia (AML), chronic lymphocytic leukemia (CLL), chronic myelogenous leukemia (CIVIL), blast phase CML, small lymphocytic lymphoma (SLL), CLL/SLL, Hodgkin lymphoma (HL), non-Hodgkin lymphoma (NHL), B-cell NHL, T-cell NHL, indolent NHL (iNHL), diffuse large B-cell lymphoma (DLBCL), mantle cell lymphoma (MCL), aggressive B-cell NHL, B-cell lymphoma (BCL), Richter's syndrome (RS), T-cell lymphoma (TCL), peripheral T-cell lymphoma (PTCL), cutaneous T-cell lymphoma (CTCL), transformed mycosis fungoides, Sezary syndrome, anaplastic large-cell lymphoma (ALCL), follicular lymphoma (FL), Waldenstrom macroglobulinemia (WM), lymphoplasmacytic lymphoma, Burkitt lymphoma, multiple myeloma (MM), amyloidosis, MPD, essential thrombocytosis (ET), myelofibrosis (MF), polycythemia vera (PV), chronic myelomonocytic leukemia (CMML), myelodysplastic syndrome (MDS), high-risk MDS, and low-risk MDS. In one embodiment, the hematologic malignancy is relapsed. In one embodiment, the hematologic malignancy is refractory. In one embodiment, the cancer or disease is in a pediatric patient (including an infantile patient). In one embodiment, the cancer or disease is in an adult patient.

It should be apparent to those skilled in the art that many additional modifications besides those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

The following non-limiting examples are included for purposes of illustration only, and are not intended to limit the scope of the range of techniques and protocols in which the compositions and methods of the present invention may find utility, as will be appreciated by one of skill in the art and can be readily implemented. The present invention has been described in terms of one or more preferred embodiments, and it should be appreciated that many equivalents, alternatives, variations, and modifications, aside from those expressly stated, are possible and within the scope of the invention.

### EXAMPLES

Targeted cancer therapies are limited by dose-limiting toxicities secondary to target engagement in non-malignant tissues. These difficulties can be minimized by targeting cancer dependencies driven by proteins with tissue- and/or tumor-restricted expression. In the following example, the inventors show that in acute myeloid leukemia (AML) cells, suppression of the myeloid-restricted p110γ-p101 axis blocks PI3K/AKT signaling, compromises cell fitness, and sensitizes to chemotherapy. Therefore, targeted inhibition of p110γ-p101 signaling selectively suppresses PI3K signaling in AML cells, and likely in cells from other myeloid malignancies, circumventing the toxicities that have historically limited clinical inhibition of PI3K.

### Materials and Methods:

### Cell Lines and Reagents

All cell lines were purchased from American Type Culture Collection (ATCC) or Duke University Cell Culture Facility (CCF) and maintained in a humidified incubator at 37° C. with 5% CO₂. The following AML cell lines were used: KG-1a, MV4; 11, OCI-AML3, Kasumi-1, OCI-AML2, Hel, THP-1, MOLM-13, NB4, NOMO-1, HL-60. All AML cell lines were cultured in RPMI-1640 medium with 10% fetal bovine serum (FBS) and 1% penicillin/streptomycin. 293FT cells were cultured in DMEM high glucose medium with 10% FBS, 1% sodium pyruvate, 1% non-essential amino acids, and 1% GlutaMax. HS-5 cells were cultured in DMEM with 10% FBS and 1% penicillin/streptomycin. MK-2206 and BYL-719 were purchased from ApexBio. All other drugs, including IPI-549, ABT-199, daunorubicin, TGX-221, CAL-101, AS252424, AS605240, were purchased from SelleckChem.

### In Vitro Drug Sensitivity Assays (GI50 Assay)

AML cells were plated in 96-well plates at a density of 5,000-10,000 cells per 100 μL per well. Wells were treated with drug dilution series at the doses indicated and cultured for 72 hours. Subsequently, relative cell viability was approximated using Promega CellTiter-Glo Luminescent Cell Viability Assay, normalized to either DMSO or the indicated background drug. GI50 values where approximated from dose-response curves plotted using GraphPad/Prism 8 software.

### Analysis of Publically-Available Gene Expression Data

Gene expression data for normal and malignant tissues was obtained from GeTex gene expression dataset and accessed via the Gepia portal. Gene dependency data was obtained from the DepMap dependency dataset. Data analyses were performed using R or GraphPad/Prism 8.

### Western Immunoblotting

Western immunoblotting was performed as previously described. Membranes were probed with primary antibodies (3-actin (13E5) (CST #4970 diluted 1:5000 in 5% BSA), p-AKT T308 (244F9) (CST #4056 diluted 1:1000 in 5% BSA), p-AKT 5473 (D9E) (CST #4060 diluted 1:1000 in 5% BSA), T-AKT (C67E7) (CST #4691 diluted 1:3000 in 5% BSA), p-GSK3(3 S9 (D85E12) (CST #5558 diluted 1:1000 in 5% BSA), p-BAD 5136 (D25H8) (CST #4366 diluted 1:1000 in 5% BSA), p-TSC2 T1462 (XX) (CST XXX), T-TSC2 (XX) (XX), p110-g (D55D5) (CST #5405 diluted 1:1000 in 5% BSA), p110α (C73F8) (CST #4249 diluted 1:1000 in 5% BSA), p110β (C33D4) (CST #3011 diluted 1:1000 in 5% BSA), p110δ (D1Q7R) (CST #34050 diluted 1:1000 in 5% BSA) or p101 (D32A5) (CST #5569 diluted 1:1000 in 5% BSA) overnight (16 hours).

### Conditioned Media and SDF1-a Cytokine Experiments

Serum free RPMI-1640 medium with incubated with a confluent plate of HS-5 cells for 36 hours. Conditioned media was centrifuged at 1200 rpm for 5 minutes and filtered with a 0.45 uM filter to remove any remaining cells. AML cells were incubated with conditioned media with or without IPI-549 for 30 minutes prior to harvesting on ice for lysate preparation. An analogous strategy was performed with SDF-1a (200 ng/mL) containing media. SDF-1a was purchased from ProteinTech and solubilized in water.

Generation of Stably Expressing Doxycycline-Inducible shRNA Cell Lines

Inducible expression of shRNAs was achieved as previously described using a doxycycline-inducible pLKO-Tet-On lentiviral system. Lentivirus was produced and cells were transduced as previously described. Following selection with puromycin, shRNA containing cells were treated with doxycycline (75 ng/mL) for 72 hours prior to analysis or experimentation. Target shRNA nucleotide sequences can be found in Table 1 below.

### Whole Genome CRISPR/Cas9 Screen

sgRNA library was amplified and prepared as previously described using the Toronto Knockout CRISPR Library—Version 3 (TKOv3) obtained from Addgene (Pooled Libraries #90294, #125517). OCI-AML2 cells were transduced at 1000× coverage of the library (72E6 cells transduced) and cultured for a minimum of 1000× coverage for the duration of the screen. After 7 days of puromycin selection, cells were divided into three treatment arms—cells treated with DMSO, IPI-549 (1 uM) or MK-2206 (1 uM)—for two weeks. All treatments were represented in each biological replicate. Genomic DNA was extracted using the QIAamp DNA Kit (Qiagen) in 25E6 cell increments. Amplification of sgRNA barcodes and indexing of each samples was performed via 2-step PCR as described elsewhere. Identification of sensitizing or resistor genes was performed as previously described by comparing the final drug-treated populations to DMSO-treated populations.

### Kaplan Meier Analysis

Survival modeling and Kaplan Meier analysis was performed on TCGA LAML data (n=200) using the R “survival” package. Overall survival (OS) was defined as the duration of time separating diagnosis from death or, in the case of survivors, the last known date of follow-up. Definition of high and low PIK3R5-expressing cohorts was made on a tertile basis.

TABLE 1
Target shRNA nucleotide sequences

| shRNA | Sequence |
|---|---|
| shPIK3CA | CCGGAATGAAAGCTCACTCTGGATTCTCGAGAATCCAGAG |
| | TGAGCTTTCATTTTTTTG (SEQ ID NO: 1) |
| shPIK3CB | CCGGGCGGGAGAGTAGAATATGTTTCTCGAGAAACATATT |
| | CTACTCTCCCGCTTTTTG (SEQ ID NO: 2) |
| shPIK3CD | CCGGGACCCAGAAGTGAACGACTTTCTCGAGAAAGTCGTT |
| | CACTTCTGGGTCTTTTTG (SEQ ID NO: 3) |
| shPIK3CG | CCGGGCAGAGCTTCTTCACCAAGATCTCGAGATCTTGGTG |
| 1 | AAGAAGCTCTGCTTTTTG (SEQ ID NO: 4) |
| shPIK3CG | CCGGGCCCTATCAAATGAAACAATTCTCGAGAATTGTTTC |
| 2 | ATTTGATAGGGCTTTTTG (SEQ ID NO: 5) |
| shPIK3CG | CCGGGCCTTATCCATTTCCCATTTACTCGAGTAAATGGGA |
| 3′UTR | AATGGATAAGGCTTTTTTG (SEQ ID NO: 6) |
| shPIK3R1 | CCGGCCTTCAGTTCTGTGGTTGAATCTCGAGATTCAACCA |
| | CAGAACTGAAGGTTTTTG (SEQ ID NO: 7) |
| shPIK3R2 | CCGGGCAGATGAAGCGTACTGCAATCTCGAGATTGCAGTA |
| | CGCTTCATCTGCTTTTTG (SEQ ID NO: 8) |
| shPIK3R3 | CCGGGCTTTGGACAACCGAGAAATACTCGAGTATTTCTCG |
| | GTTGTCCAAAGCTTTTTG (SEQ ID NO: 9) |
| shPIK3R5 | CCGGCAGGATCTATAAACTCTTCAACTCGAGTTGAAGAGT |
| | TTATAGATCCTGTTTTTG (SEQ ID NO: 10) |
| shPIK3R6 | CCGGCCAGATCTACACAGTCAAGATCTCGAGATCTTGACT |
| | GTGTAGATCTGGTTTTTG (SEQ ID NO: 11) |

### Results

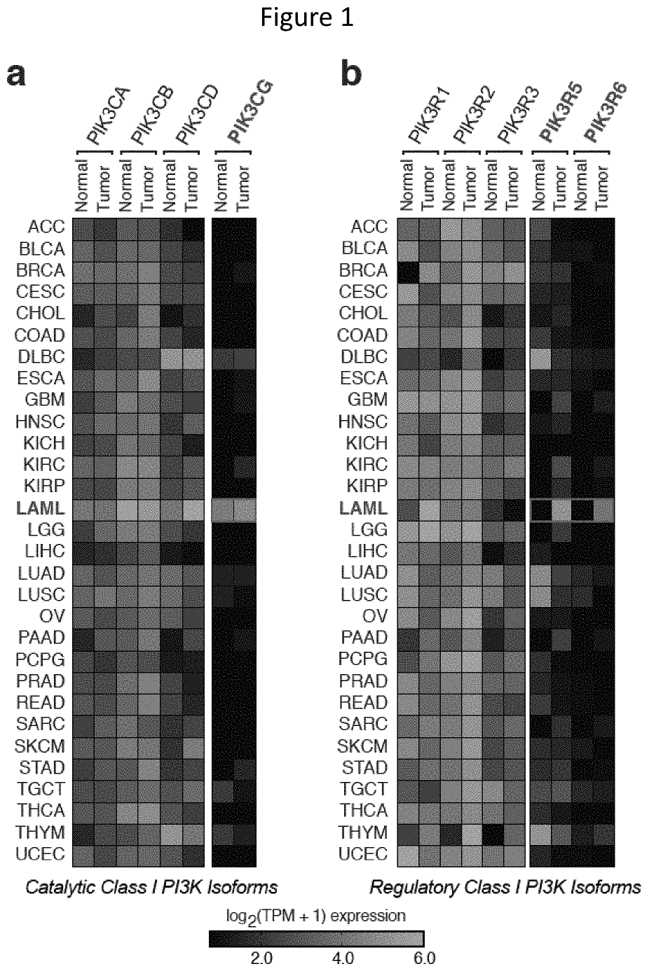

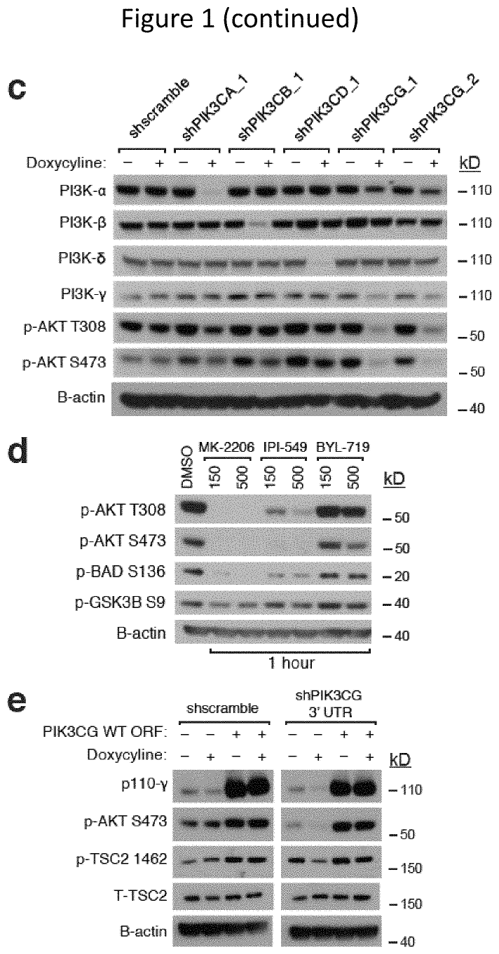

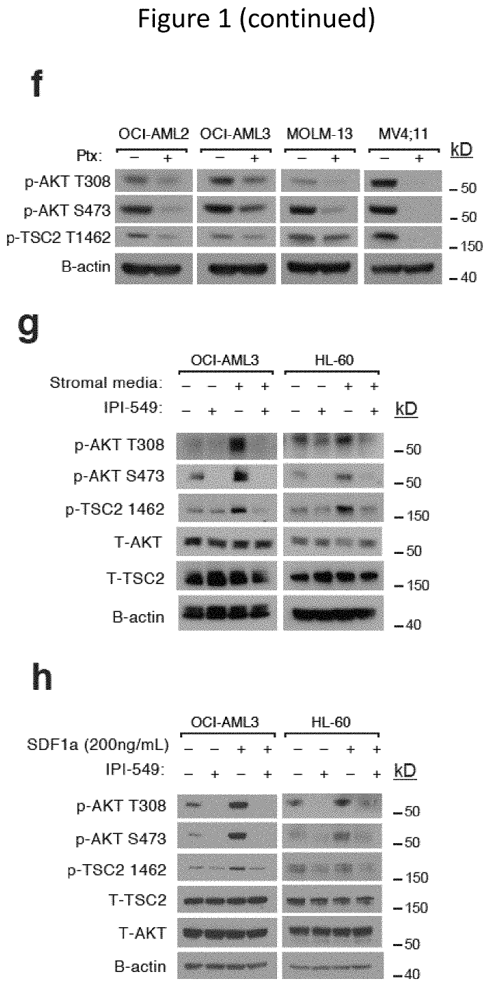

There are four class I PI3K catalytic isoforms, three of which (PIK3CA, PIK3CB and PIK3CD, encoding p110α, p110β, and p110δ, respectively) display near-ubiquitous expression across diverse human tissue types, in both the malignant and non-malignant settings (FIG. 1a). By contrast, expression of PIK3CG (encoding p110γ) is largely restricted to cells of the hematopoietic system, and in particular, the myeloid compartment (FIG. 1a). The limited expression of p110γ is complemented by the expression of its exclusive, cognate regulatory subunits, PIK3R5 and PIK3R6 (encoding p101 and p87, respectively), both of which are upregulated in AML (FIG. 1b).

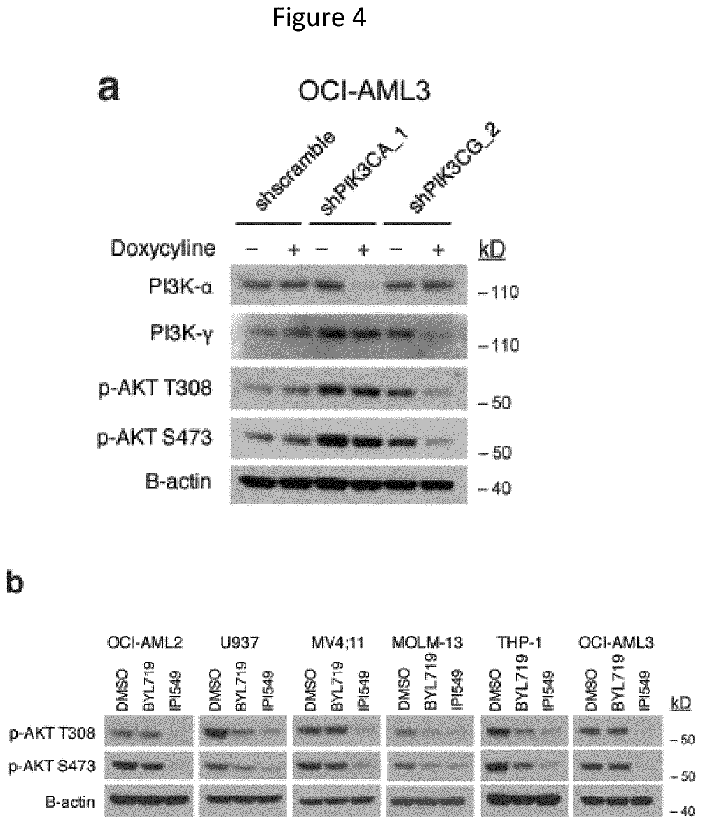

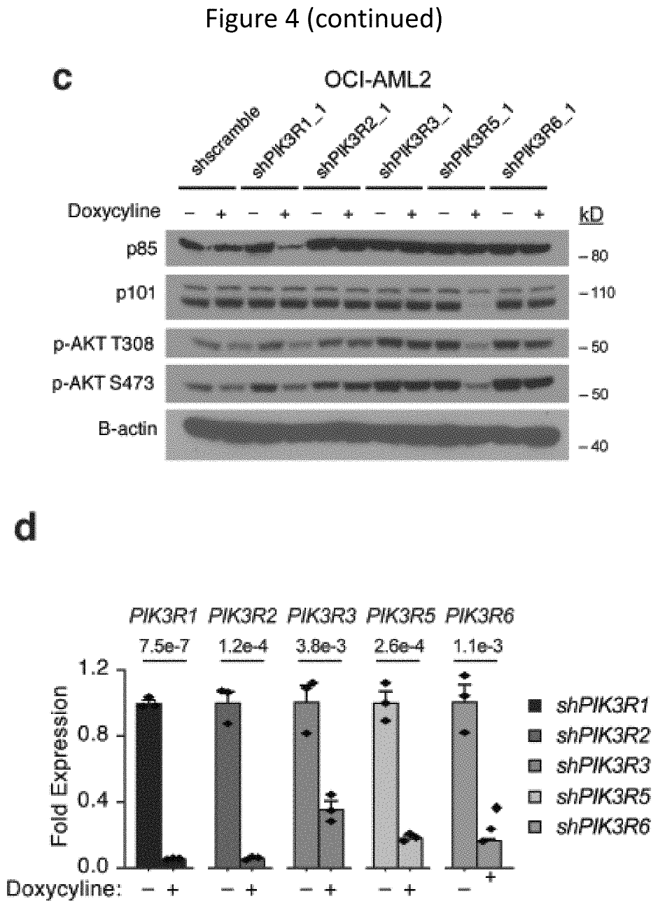

First, we asked if the highly tissue-specific expression pattern of the p110γ-p101-p87 signaling network in AML conferred isoform-specific control of downstream AKT signaling. Doxycycline(dox)-inducible short-hairpin RNA (shRNA) mediated knockdown of PIK3CA, PIK3CB and PIK3CD had no effect on activating phosphorylation marks on AKT, at both the Thr308 and Ser473 sites (FIG. 1c, FIG. 4a). Conversely, shRNAs targeting PIK3CG resulted in loss of AKT phosphorylation at both residues (FIG. 1c, FIG. 4a). This finding was recapitulated using isoform-selective inhibitors of PI3K. Phosphorylation of AKT and its downstream substrates was lost in response to small-molecule inhibition of p110γ with IPI-549, while selective inhibition of p110α with BYL-719 (Alpelisib) was minimally capable of suppressing AKT pathway phosphorylation across cell line models of AML (FIG. 1d, FIG. 4b). Conversely, we found that overexpression of wild-type PIK3CG under a constitutive EF1a promotor resulted in hyperactivation of AKT signaling (FIG. 1e). Importantly, re-introduction of wild-type PIK3CG was able to fully rescue AKT suppression secondary to a dox-inducible shRNA against the 3′ UTR region of PIK3CG (FIG. 1e). These data indicate that p110γ is both necessary and sufficient for PI3K/AKT signaling in AML.

Given the known association of p110γ with both p101 and p8′7, we wondered which of the two regulatory subunits influenced activation of AKT. Interestingly, shRNAs targeting the regulatory subunits of class I PI3K isoforms identified depletion of only PIK3R5, and not PIK3R6, to modulate AKT phosphorylation (FIG. 4c,d). Prior studies in inflammatory cells [12, 17] have resolved key differences between the upstream drivers of p110γ-p101 and p110γ-p87 signaling. Namely, p110γ-p87 is downstream of both GPCRs and Ras/RTKs while p110γ-p101 is driven exclusively by GPCRs. Therefore, we hypothesized that the baseline AKT signaling observed in AML cells originates from GPCRs and is transduced by p110γ-p101 to activate AKT. Consistent with this notion, we observed diminished AKT phosphorylation in AML cell lines treated with pertussis toxin (Ptx), a general uncoupler of GPCR—heterotrimeric G-protein complexes (FIG. 1f). G-protein coupled chemokine receptors play a key role in supporting leukemic cell proliferation, migration and chemoresistance through activation of mitogenic signaling axes that include PI3K/AKT [18, 19]. Soluble factors secreted by the surrounding bone marrow stroma or by the malignant cells themselves provide the requisite ligands for chemokine receptor engagement [20]. We hypothesized that stroma-derived activation of PI3K/AKT signaling may be dependent upon p110γ and could therefore be quenched by p110γ inhibition. To test this, AML cells were incubated with serum-free media that was either unconditioned or conditioned with human bone marrow mesenchymal stromal cells (BM-MSCs). We observed strong activation of AKT signaling in cells incubated with conditioned media relative to those incubated with unconditioned media. This effect was fully precluded by treatment with IPI-549, indicating that the capacity of the stroma-conditioned milieu to activate AKT signaling in AML cells relies on p110γ (FIG. 1g). Relatedly, we sought to investigate the p110γ-dependency of CXCL12 (SDF1α)—CXCR4 signaling, which has been widely characterized as a mediator of stroma-dependent survival signaling in leukemia [18, 21-23]. To test if SDF1a activated PI3K/AKT through p110γ, we incubated serum-deprived AML cells with and without SDF1α in the presence or absence of IPI-549. We found that stimulation with SDF1a activated AKT, and that this activation could also be prevented by IPI-549 treatment, suggesting that SDF1α-dependent AKT signaling in AML requires p110γ (FIG. 1h). Taken together, these findings point to an important role for p110γ in communicating cell survival and growth signals from the tumor microenvironment.

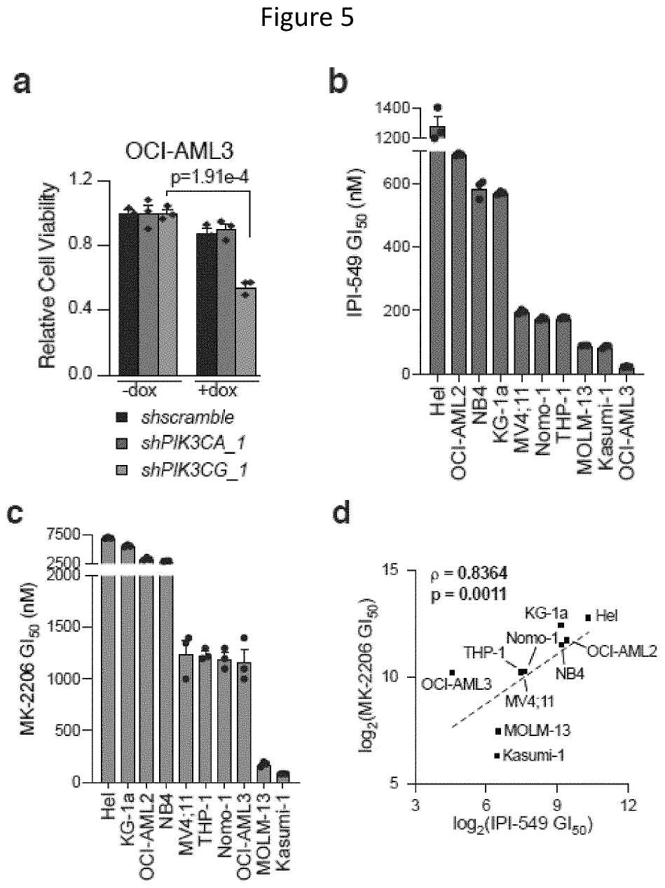

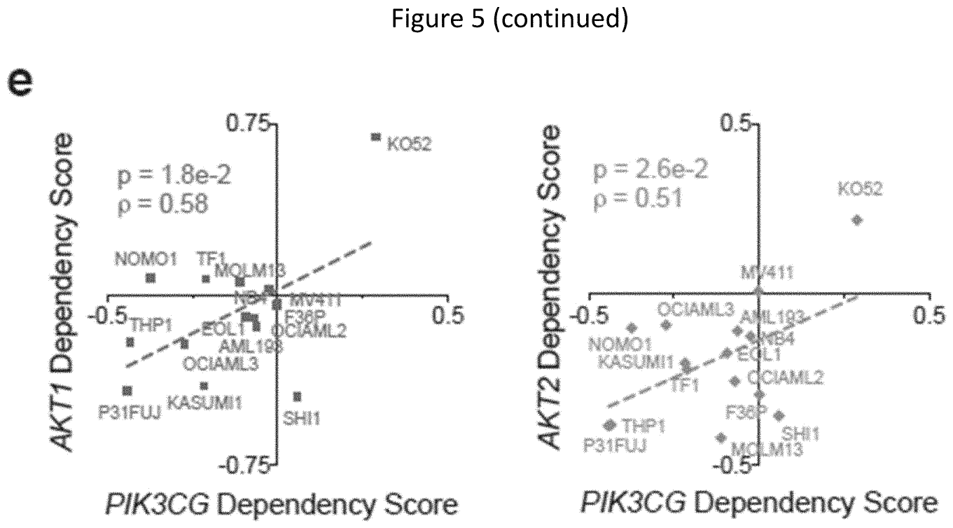

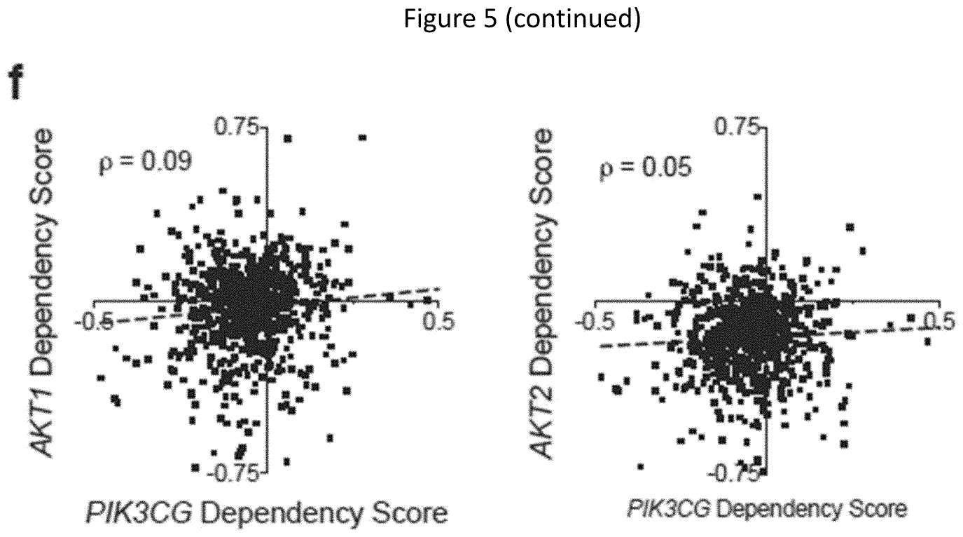

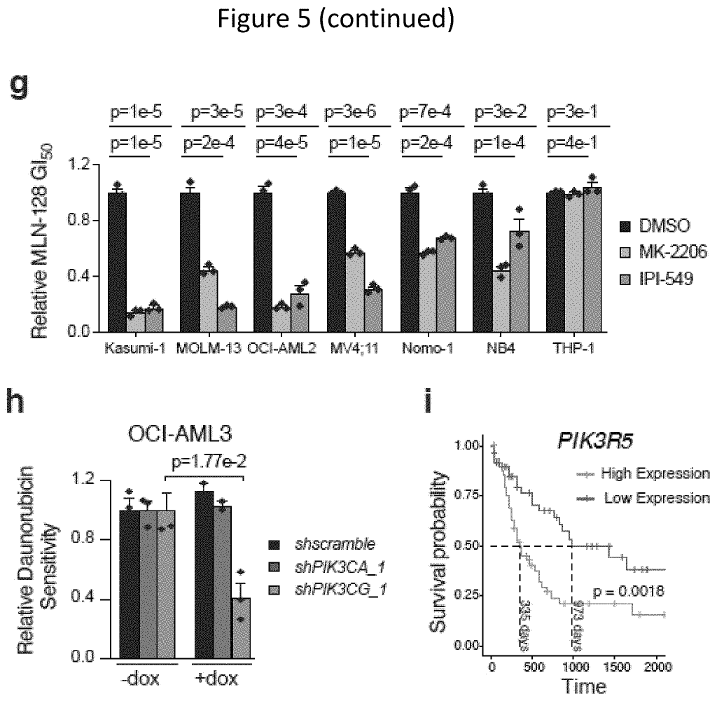

In light of the dominant role of p110γ on AKT signaling in AML, we suspected that, among PI3K isoforms, p110γ alone would be important for cell fitness and that this importance would be specific to AML. Indeed, in a publicly available genetic dependency dataset [24], PIK3CG was the only PI3K isoform essential to AML cell lines (FIG. 2a). Further, PIK3CG was most essential in AML (and secondarily in ALL) with minimal essentiality in malignancies of non-hematopoietic origin. By contrast, PIK3CA and PIK3CB were essential across malignancies of diverse tissue types (FIG. 2a). Similarly, PIK3R5 was essential only in AML and CLL, both myeloid malignancies, while the regulatory subunits associated with p110α, p110β, and p110δ exhibited widespread essentiality across cancers of other tissues (PIK3R6 is not included in dataset) (FIG. 2a). These data, in combination with the restricted expression pattern of PIK3CG (FIG. 1a), support the notion that p110γ is dispensable for AKT activity in non-hematopoietic tissues but vital to the pro-survival functions of AKT in AML. In agreement with the analyzed dataset, AML cell viability was reduced following shRNA-mediated depletion of PIK3CG but not with shRNAs targeting the other catalytic members of the PI3K family (FIG. 2b, FIG. 5a).

We then reasoned that AML cells should exhibit p110γ dependence proportional to their AKT dependence. We tested this in a panel of 10 AML cell lines and found that sensitivity to p110γ inhibition with IPI-549 was strongly correlated with sensitivity to AKT inhibition with MK-2206 (FIG. 5b-d). Further, PIK3CG dependency was significantly correlated with dependency on AKT1 and AKT2 in AML cell lines but not in cell lines from other tissues (FIG. 2e,f). To compare the genetic determinants of AKT and p110γ dependency more broadly, we conducted full-genome CRISPR/Cas9-based loss-of-function screens in OCI-AML2 cells continuously cultured in either MK-2206 or IPI-549 for 12 days. We observed significant correlation between the genetic modifiers of sensitivity to the two drugs, with substantial overlap amongst the most enriched and most depleted sgRNA constructs (FIG. 2c). Collectively, these data demonstrate that the genetic mediators of sensitivity to AKT and p110γ inhibition are highly shared, consistent with our data positioning p110γ as the major regulator of AKT signaling (FIG. 1c,d) in AML.

Given the well-established role of PI3K signaling as a driver of resistance to cytotoxic and targeted chemotherapies [25-27], we asked whether the anti-tumor effects of p110γ inhibition we observed in AML could be leveraged to potentiate the effect of other therapies. Our CRISPR/Cas9 drug-modifier screens identified a common set of genes that significantly altered sensitivity to both MK-2206 and IPI-549. Cells harboring sgRNAs targeting BCL2 were most depleted in both MK-2206 and IPI-549 treated screens, while cells harboring sgRNAs targeting negative regulators of mTOR signaling (TSC1, TSC2, NPRL2) were most enriched in both screens (FIG. 2c). These findings are consistent with reports positioning BCL2 and mTOR as targets whose inhibition synergistically enhances PI3K pathway ablation [28, 29]. We validated these interactions using the BCL2-inhibitor, ABT-199 (Venetoclax), or the ATP-competitive mTOR inhibitor, MLN-128, both of which enhanced the potency of AKT/p110γ inhibition across a panel of AML cell lines (FIG. 2d, FIG. 5g). Pairing venetoclax with p110γ inhibition is especially attractive, given the recent successes of venetoclax-based regimens in the treatment of AML [30, 31] and the robust sensitization effect across all cell line models tested. We also paired p110γ suppression with daunorubicin, an anthracycline that forms the backbone of induction therapy in AML [32, 33]. In 9/10 AML cell lines tested, p110γ inhibition significantly potentiated daunorubicin sensitivity while p110α inhibition did not alter sensitivity to daunorubicin in any of the models tested (FIG. 2e). Further, genetic ablation of PIK3CG and not the other catalytic isoforms of PI3K increased the potency of daunorubicin (FIG. 2f, Extended FIG. 2h). Parenthetically, we noted that AML patients with low PIK3R5 expression achieved a significantly longer median overall survival than patients with high PIK3R5 expression, according to data from the TCGA [34] (FIG. 5i). This suggests that hyperactivation of the p110γ-p101 signaling axis through PIK3R5 upregulation may drive aggressiveness and resistance to anthracyclines in AML.

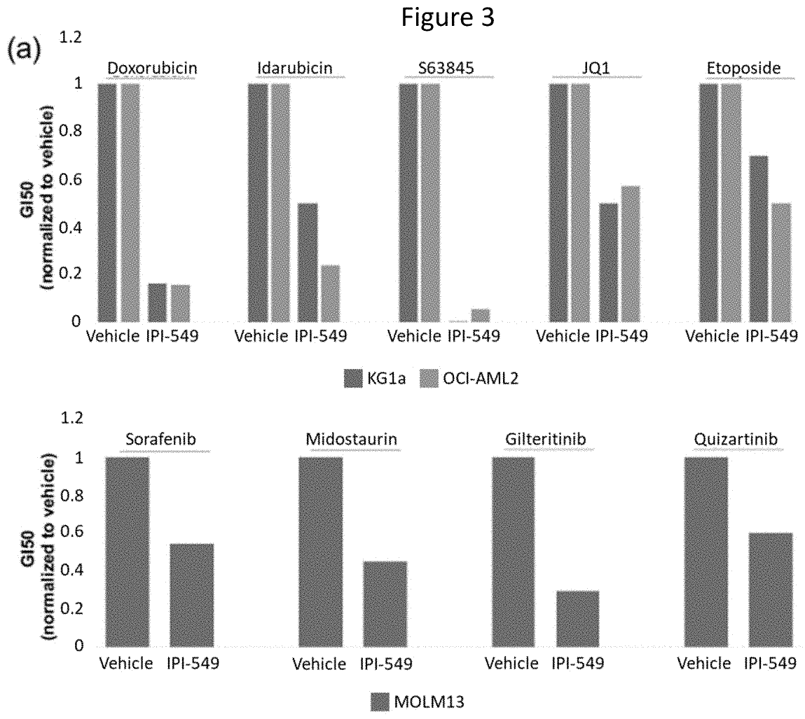

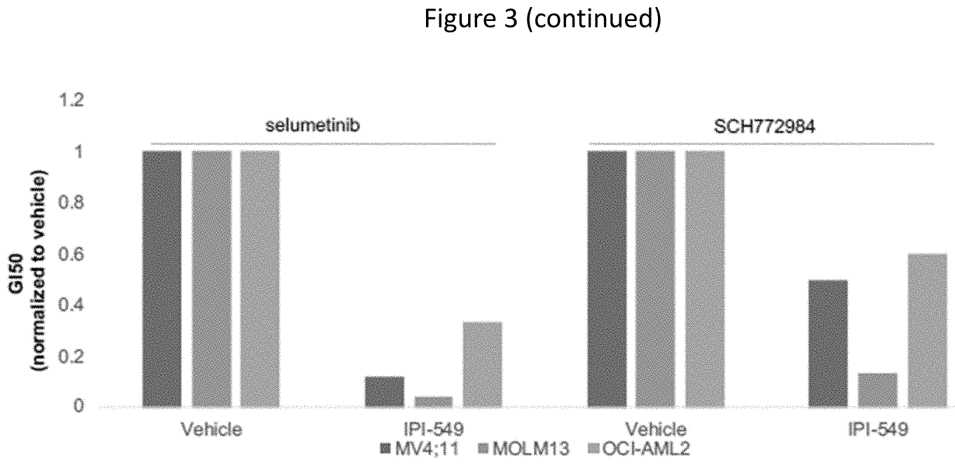

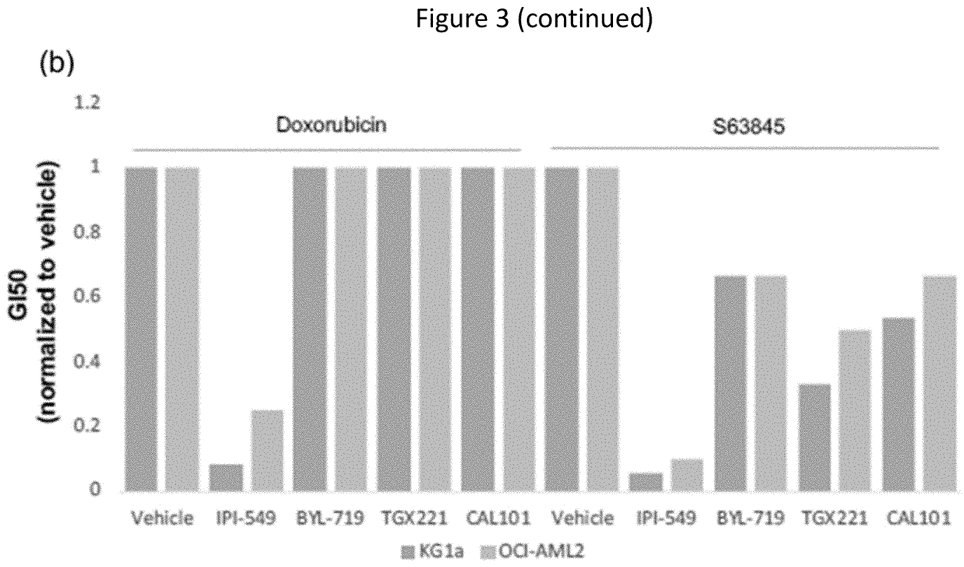

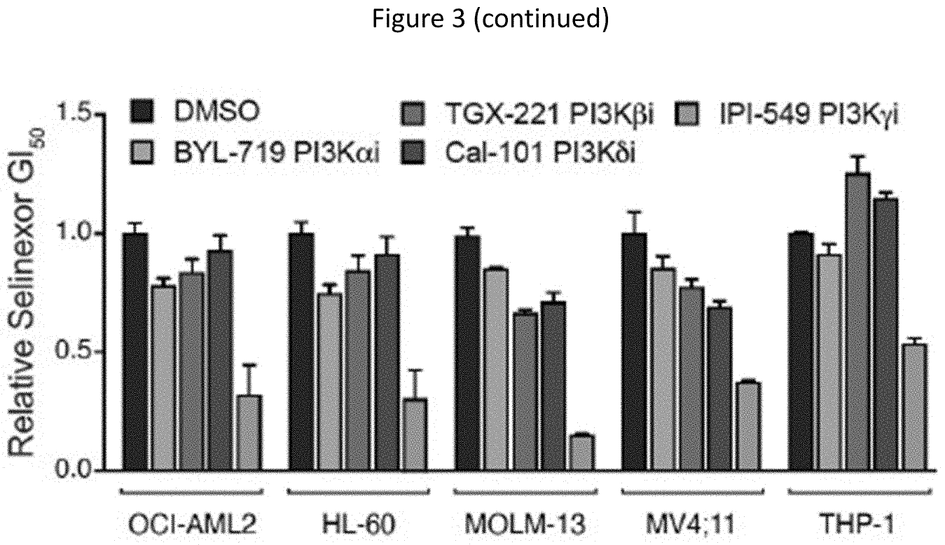

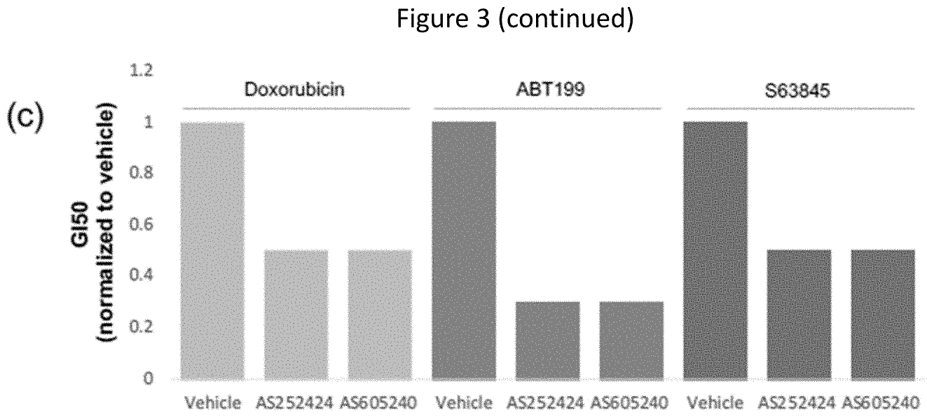

To more broadly define the approved and emerging chemotherapeutic agents to which AML cells can be sensitized through PI3Kγ inhibition, we performed additional studies which revealed that IPI-549 treatment can sensitize AML cells to the anthracyclines doxorubicin and idarubicin (in addition to daunorubicin, shown above), MCL-1 inhibition with 563845, FLT-3 inhibition with sorafenib, midostaurin, gilteritinib, and quizartinib, MEK1/2 or ERK1/2 inhibition with selumetinib or SCH772984, the topoisomerase inhibitor etoposide, the BET inhibitor JQ1, and the XPO1/CRM1 inhibitor selinexor (FIG. 3a-b). These effects are not observed with specific inhibitors of the alternative PI3K catalytic isoforms, p110α, p110β, and p110δ (using BYL-719, TGX-221, and CAL-101, respectively) (FIG. 3b), and similar chemosensitization to doxorubicin, ABT199, and S63845 was observed with the structurally distinct, p110γ selective inhibitors AS252424 and AS605240 (FIG. 3c). Finally, chemosensitization mediated by PI3Kγ inhibition with IPI-549 is not observed with all drugs relevant to AML; specifically, it was not observed with the antimetabolites/nucleoside analogs cytarabine, azacytidine, and decitabine, the antifolate methotrexate, the hedgehog pathway inhibitor glasdegib, the taxane docetaxel, and the platinum drug oxaliplatin (FIG. 6).

Together, these data advance isoform-specific inhibition of p110γ as a therapeutic strategy to suppress AKT signaling in AML and other myeloid malignancies while sparing the PI3K activity vital to maintaining nutrient homeostasis in other tissues. The findings presented here argue for cancer therapeutics specifically designed to target dependencies driven by proteins with tissue- and/or tumor-restricted expression. This approach is not without precedent; selective inhibition of p110δ, a critical signaling mediator in lymphocytes, has shown impressive clinical efficacy in B-cell malignancies and is now FDA-approved in combination with rituximab in relapsed/refractory CLL [35, 36]. One could envision that a highly selective p110γ inhibitor may similarly be used as an adjuvant therapy in AML. Suppressing such “lineage-addictions” [37, 38] could permit directed targeting of the therapy to the tissue compartment harboring malignancy, establishing the grounds for a therapeutic window.

### REFERENCES

1 Fruman, D. A., et al., The PI3K Pathway in Human Disease. Cell, 2017. 170(4): p. 605-635.
2. Bilanges, B., Y. Posor, and B. Vanhaesebroeck, PI3K isoforms in cell signalling and vesicle trafficking. Nat Rev Mol Cell Biol, 2019. 20(9): p. 515-534.
3. Janku, F., T. A. Yap, and F. Meric-Bernstam, Targeting the PI3K pathway in cancer: are we making headway? Nat Rev Clin Oncol, 2018. 15(5): p. 273-291.
4. Millis, S. Z., et al., Landscape of Phosphatidylinositol-3-Kinase Pathway Alterations Across 19784 Diverse Solid Tumors. JAMA Oncol, 2016. 2(12): p. 1565-1573.
5. Andre, F., et al., Alpelisib for PIK3CA-Mutated, Hormone Receptor Positive Advanced Breast Cancer. N Engl J Med, 2019. 380(20): p. 1929-1940.
6. Baselga, J., et al., Phase III study of taselisib (GDC-0032)+fulvestrant (FULV) v FULV in patients (pts) with estrogen receptor (ER) positive, PIK3CA-mutant (MUT), locally advanced or metastatic breast cancer (MBC): Primary analysis from SANDPIPER. Journal of Clinical Oncology, 2018. 36(18).
7 Mateo, J., et al., A First-Time-in-Human Study of GSK2636771, a Phosphoinositide 3 Kinase Beta-Selective Inhibitor, in Patients with Advanced Solid Tumors. Clin Cancer Res, 2017. 23(19): p. 5981-5992.
8. Maira, S. M., et al., Identification and characterization of NVP-BKM-120, an orally available pan-class I PI3-kinase inhibitor. Mol Cancer Ther, 2012. 11(2): p. 317-28.
9. Maira, S. M., et al., Identification and characterization of NVP-BEZ235, a new orally available dual phosphatidylinositol 3-kinase/mammalian target of rapamycin inhibitor with potent in vivo antitumor activity. Mol Cancer Ther, 2008. 7(7): p. 1851-63.
10. Lannutti, B. J., et al., CAL-101, a p110delta selective phosphatidylinositol-3-kinase inhibitor for the treatment of B-cell malignancies, inhibits PI3K signaling and cellular viability. Blood, 2011. 117(2): p. 591-4.
11. Fritsch, C., et al., Characterization of the novel and specific PI3Kalpha inhibitor NVP-BYL719 and development of the patient stratification strategy for clinical trials. Mol Cancer Ther, 2014. 13(5): p. 1117-29.
12. Kaneda, M. M., et al., PI3Kgamma is a molecular switch that controls immune suppression. Nature, 2016. 539(7629): p. 437-442.
13. Hirai, H., et al., MK-2206, an allosteric Akt inhibitor, enhances antitumor efficacy by standard chemotherapeutic agents or molecular targeted drugs in vitro and in vivo. Mol Cancer Ther, 2010. 9(7): p. 1956-67.
14. Di Leo, A., et al., Buparlisib plus fulvestrant in postmenopausal women with hormone-receptor-positive, HER2-negative, advanced breast cancer progressing on or after mTOR inhibition (BELLE-3): a randomised, double-blind, placebo-controlled, phase 3 trial. Lancet Oncol, 2018. 19(1): p. 87-100.
15. Krop, I. E., et al., Pictilisib for oestrogen receptor-positive, aromatase inhibitor-resistant, advanced or metastatic breast cancer (FERGI): a randomised, double-blind, placebo-controlled, phase 2 trial. Lancet Oncol, 2016. 17(6): p. 811-821.
16. Hopkins, B. D., et al., Suppression of insulin feedback enhances the efficacy of PI3K inhibitors. Nature, 2018. 560(7719): p. 499-503.
17. Schmid, M. C., et al., Receptor tyrosine kinases and TLR/IL1Rs unexpectedly activate myeloid cell PI3kgamma, a single convergent point promoting tumor inflammation and progression. Cancer Cell, 2011. 19(6): p. 715-27.
18. Carter, B. Z., et al., Anti-apoptotic ARC protein confers chemoresistance by controlling leukemia-microenvironment interactions through a NFkappaB/IL1beta signaling network. Oncotarget, 2016. 7(15): p. 20054-67.
19. Burkle, A., et al., Overexpression of the CXCR5 chemokine receptor, and its ligand, CXCL13 in B-cell chronic lymphocytic leukemia. Blood, 2007. 110(9): p. 3316-25.
20. Duarte, D., E. D. Hawkins, and C. Lo Celso, The interplay of leukemia cells and the bone marrow microenvironment. Blood, 2018. 131(14): p. 1507-1511.
21. Azab, A. K., et al., CXCR4 inhibitor AMD3100 disrupts the interaction of multiple myeloma cells with the bone marrow microenvironment and enhances their sensitivity to therapy. Blood, 2009. 113(18): p. 4341-51.
22. Zeng, Z., et al., Targeting the leukemia microenvironment by CXCR4 inhibition overcomes resistance to kinase inhibitors and chemotherapy in AML. Blood, 2009. 113(24): p. 6215-24.
23. Uy, G. L., et al., A phase 1/2 study of chemosensitization with the CXCR4 antagonist plerixafor in relapsed or refractory acute myeloid leukemia. Blood, 2012. 119(17): p.
24. Meyers, R. M., et al., Computational correction of copy number effect improves specificity of CRISPR-Cas9 essentiality screens in cancer cells. Nat Genet, 2017. 49(12): p. 1779-1784.
25. Sampath, D., et al., Pharmacodynamics of cytarabine alone and in combination with 7-hydroxystaurosporine (UCN-01) in AML blasts in vitro and during a clinical trial. Blood, 2006. 107(6): p. 2517-24.
26. Schmid, P., et al., Capivasertib Plus Paclitaxel Versus Placebo Plus Paclitaxel As First-Line Therapy for Metastatic Triple-Negative Breast Cancer: The PAKT Trial. J Clin Oncol, 2020. 38(5): p. 423-433.
27. Jones, R. H., et al., Capivasertib (AZD5363) plus fulvestrant versus placebo plus fulvestrant after relapse or progression on an aromatase inhibitor in metastatic ER-positive breast cancer (FAKTION): A randomized, double-blind, placebo-controlled, phase II trial. Journal of Clinical Oncology, 2019. 37(15).
28. Sathe, A., et al., Parallel PI3K, AKT and mTOR inhibition is required to control feedback loops that limit tumor therapy. PLoS One, 2018. 13(1): p. e0190854.
29. Pham, L. V., et al., Strategic Therapeutic Targeting to Overcome Venetoclax Resistance in Aggressive B-cell Lymphomas. Clinical Cancer Research, 2018. 24(16): p. 3967-3980.
30. Pollyea, D. A., et al., Venetoclax with azacitidine disrupts energy metabolism and targets leukemia stem cells in patients with acute myeloid leukemia. Nat Med, 2018. 24(12): p. 1859-1866.
31. DiNardo, C. D., et al., Safety and preliminary efficacy of venetoclax with decitabine or azacitidine in elderly patients with previously untreated acute myeloid leukaemia: a non-randomised, open-label, phase 1b study. Lancet Oncol, 2018. 19(2): p. 216-228.
32. Dohner, H., et al., Diagnosis and management of AML in adults: 2017 ELN recommendations from an international expert panel. Blood, 2017. 129(4): p. 424-447.
33. Dohner, H., D. J. Weisdorf, and C. D. Bloomfield, Acute Myeloid Leukemia. N Engl J Med, 2015. 373(12): p. 1136-52.
34. Cancer Genome Atlas Research, N., et al., The Cancer Genome Atlas Pan-Cancer analysis project. Nat Genet, 2013. 45(10): p. 1113-20.
35. Furman, R. R., et al., Idelalisib and rituximab in relapsed chronic lymphocytic leukemia. N Engl J Med, 2014. 370(11): p. 997-1007.
36. Gopal, A. K., et al., PI3Kdelta inhibition by idelalisib in patients with relapsed indolent lymphoma. N Engl J Med, 2014. 370(11): p. 1008-18.
37. Garraway, L. A. and W. R. Sellers, Lineage dependency and lineage-survival oncogenes in human cancer. Nat Rev Cancer, 2006. 6(8): p. 593-602.
38. Vias, M., A. Ramos-Montoya, and I. G. Mills, Terminal and progenitor lineage-survival oncogenes as cancer markers. Trends Mol Med, 2008. 14(11): p. 486-94.

1. A method of reducing the viability of a cell that expresses PI3Kγ in a subject, the method comprising administering to the subject a therapeutically effective amount of a PI3Kγ inhibitor.

2. The method of claim 1, wherein the subject has a myeloid malignancy.

3. The method of claim 2, wherein the myeloid malignancy is a myeloproliferative neoplasm (MPN) or a myelodysplastic syndrome (MDS).

4. The method of claim 2, wherein the myeloid malignancy is acute myeloid leukemia.

5. The method of claim 2, the method further comprising administering to the subject a therapeutically effective amount of a chemotherapeutic agent in combination with the PI3Kγ inhibitor.

6. A method of treating a myeloid malignancy in a subject, the method comprising administering to the subject a therapeutically effective amount of a PI3Kγ inhibitor.

7. The method of claim 6, wherein the myeloid malignancy is acute myeloid leukemia.

8. The method of claim 7, further comprising administering a therapeutically effective amount of a chemotherapeutic agent.

9. The method of claim 8, wherein the chemotherapeutic agent is selected from the group consisting of: a BCL2-inhibitor, an mTOR inhibitor, an anthracycline, a MCL-1 inhibitor, a FLT-3 inhibitor, an MEK1/2 inhibitor, an ERK1/2 inhibitor, a topoisomerase inhibitor, a BET inhibitor, or a XPOl/CRMl inhibitor.

10. The method of claim 9, wherein the chemotherapeutic agent is selected from the group consisting of: ABT-199, MLN-128, daunorubicin, doxorubicin, idarubicin, 563845, sorafenib, midostaurin, gilteritinib, quizartinib, selumetinib, SCH772984, etoposide, JQ1, and selinexor.

11. A method of sensitizing a cell that expresses PI3Kγ in a subject to a chemotherapeutic agent, the method comprising administering to the subject a therapeutically effective amount of a PI3Kγ inhibitor and a therapeutically effective amount of a chemotherapeutic agent.

12. The method of claim 11, wherein the method further comprises determining whether the myeloid cell expresses PI3Kγ prior to the administration step.

13. The method of claim 11, wherein the myeloid cell is associated with a myeloid malignancy.

14. The method of claim 13, wherein the myeloid malignancy is a myeloproliferative neoplasm (MPN) or a myelodysplastic syndrome (MDS).

15. The method of claim 13, wherein the myeloid malignancy is acute myeloid leukemia.

16. The method of claim 11, wherein the chemotherapeutic agent is proapoptotic.

17. The method of claim 11, wherein the chemotherapeutic agent is selected from the group consisting of: a BCL2-inhibitor, an mTOR inhibitor, an anthracycline, a MCL-1 inhibitor, a FLT-3 inhibitor, an MEK1/2 inhibitor, an ERK1/2 inhibitor, a topoisomerase inhibitor, a BET inhibitor, or a XPOl/CRMl inhibitor.

18. The method of claim 17, wherein the chemotherapeutic agent is selected from the group consisting of: ABT-199, MLN-128, daunorubicin, doxorubicin, idarubicin, 563845, sorafenib, midostaurin, gilteritinib, quizartinib, selumetinib, SCH772984, etoposide, JQ1, and selinexor.

19. The method of claim 11, wherein the PI3Kγ inhibitor is IPI-549, AS252424, or AS605240.

20. The method of claim 8, wherein the myeloid malignancy is resistant to at least one chemotherapeutic agent.
